(19) United States
(12) Patent Application Publication
(10) Pub. No.: US 2021/0038254 A1
(43) Pub. Date: Feb. 11, 2021

(54) Cannulation Apparatus and Method

(71) Applicant: Avent, Inc., Alpharetta, GA (US)

(72) Inventor: Karl Thomas Bjurbo, Cumming, GA (US)

(21) Appl. No.: 16/531,478

(22) Filed: Aug. 5, 2019

(51) Int. Cl. A61B 17/34; A61F 5/00; A61B 17/00

(52) U.S. Cl. CPC A61B 17/3421; A61F 5/0079; A61B 2017/00818; A61B 2017/00278; A61B 17/00234

(57) ABSTRACT

Cannulation devices and methods are provided. For example, a cannulation device comprises a cannula for disposition within a patient's body and having a distal end with threads disposed at the distal end. In some embodiments, the device is a pyloric sphincter cannulation device comprises a cannula for positioning within an intestine of a patient's body. The cannula has a distal end and threads are disposed at the distal end. The threads are configured to be rotated when disposed adjacent the patient's pyloric sphincter. A method for traversing a pyloric sphincter of a patient may include inserting a cannulation device into the patient's gastrointestinal tract; ascertaining a distal end of the cannulation device is adjacent the pyloric sphincter; rotating threads of the cannulation device into the pyloric sphincter; and advancing the cannulation device through the pyloric sphincter.

### FIELD

The present subject matter relates generally to a cannulation system, method, and apparatus and, more particularly, to such systems, methods, and apparatus for cannulation of the pyloric sphincter.

### BACKGROUND

Physicians and other health care providers frequently use catheters or cannulas, which include tubes inserted into the human body, to treat patients. Some such tubes, such as feeding tubes or the like, are types of tubes that are placed in the gastrointestinal tract for patients experiencing a variety of ailments. For example, nasogastric (NG) tubes are placed through the nasal cavity and usually are intended to traverse through the esophagus down into the stomach and into the small bowel or intestine. To pass from the stomach to the small intestine, the tube must be inserted through the pyloric sphincter, a type of muscular valve between the stomach and the small intestine that is usually closed but periodically dilates to permit the stomach's contents to flow from the stomach to the small intestine. Typically, a healthcare provider must wait for the natural dilation of the pyloric sphincter to insert the tube therethrough. However, determining whether the pyloric sphincter has sufficiently dilated to admit the tube can be challenging and it can be time-consuming to wait for dilation of the pyloric sphincter to insert the tube. Accordingly, improved cannulation devices and methods that overcome such shortcomings would be beneficial.

### SUMMARY

Objects and advantages of the invention will be set forth in part in the following description, or may be obvious from the description, or may be learned through practice of the invention.

In one aspect, the present subject matter is directed to a cannulation device. The cannulation device comprises a cannula for disposition within a patient's body and having a distal end. The cannulation device further comprises threads disposed at the distal end. It should also be understood that the device may further include any of the additional features as described herein.

In another aspect, the present disclosure is directed to a pyloric sphincter cannulation device. The pyloric sphincter cannulation device comprises a cannula for positioning within an intestine of a patient's body. The cannula has a distal end and threads are disposed at the distal end. The threads are configured to be rotated when the threads are disposed adjacent the patient's pyloric sphincter. It should also be appreciated that the pyloric sphincter cannulation device may further include any of the additional features as described herein.

In yet another aspect, the present disclosure is directed to a method for traversing a pyloric sphincter of a patient. The method comprises inserting a cannulation device into the patient's gastrointestinal tract; ascertaining a distal end of the cannulation device is adjacent the pyloric sphincter; rotating threads of the cannulation device into the pyloric sphincter; and advancing the cannulation device through the pyloric sphincter. It should also be understood that the method may further include any of the additional features as described herein.

These and other features, aspects and advantages of the present subject matter will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present subject matter, including the best mode thereof, directed to one of ordinary skill in the art, is set forth in the specification, which makes reference to the appended figures, in which:

FIG. 1 provides a schematic view of a portion of a patient's gastrointestinal tract, including a stomach, a pylorus having a pyloric sphincter, and a duodenum, according to an exemplary embodiment of the present subject matter.

FIG. 2 provides a schematic view of a cannulation device for cannulation of the pyloric sphincter, according to an exemplary embodiment of the present subject matter.

FIG. 3 provides a schematic view of a cannulation device for cannulation of the pyloric sphincter, according to another exemplary embodiment of the present subject matter.

FIG. 4 provides a schematic view of a cannulation device for cannulation of the pyloric sphincter, according to still another exemplary embodiment of the present subject matter.

FIG. 5 provides a schematic view of a cannulation device for cannulation of the pyloric sphincter, according to yet another exemplary embodiment of the present subject matter.

FIG. 6 provides a flow diagram of a method for cannulating a pyloric sphincter, according to an exemplary embodiment of the present subject matter.

### DETAILED DESCRIPTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Generally, the present subject matter provides a cannulation device, e.g., for cannulating sphincters such as pyloric sphincters. More particularly, the present subject matter provides a screw mechanism on a tip of a cannula such as a feeding tube to simplify sphincter cannulation. For instance, the cannula or feeding tube tip may be equipped with a rubber screw mechanism. When the tip of the rubber screw is located by or at the pyloric, then the screw can be rotated, e.g., manually or mechanically. The rotation of the screw allows the cannula to transverse the sphincter, thereby cannulating the sphincter. Similarly, the screw may be rotated to reverse the path of the cannula; that is, the screw mechanism may be used to back the cannula out through the sphincter, e.g., for the reverse of cannulate. Further, the present subject matter provides systems and methods for traversing a sphincter, such as a pyloric sphincter. It will be appreciated that, more generally, the present subject matter also may be used to, e.g., propel a cannula through a patient's body, such as through the patient's stomach or esophagus, by rotating the screw mechanism.

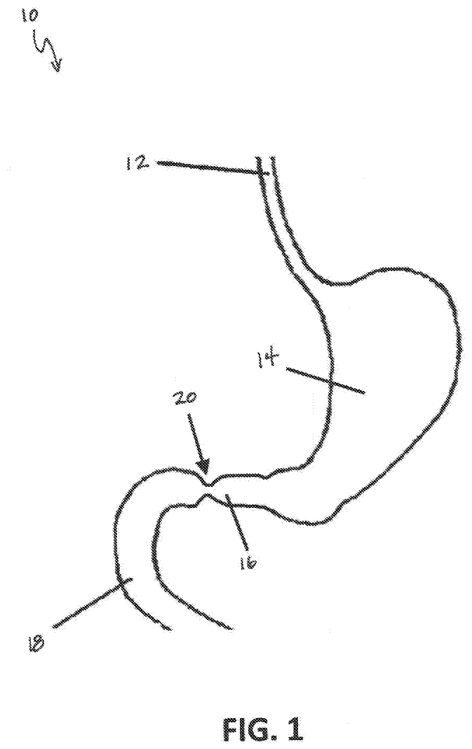

Referring now to the drawings, wherein identical numerals indicate the same elements throughout the figures, FIG. 1 provides a schematic view of a portion of a patient's gastrointestinal or digestive tract. The portion of the gastrointestinal tract 10 shown in the exemplary embodiment of FIG. 1 includes an esophagus 12, a stomach 14, a pylorus 16, and a duodenum 18 of the patient. Further, as shown in FIG. 1, the pylorus 16 includes a pyloric sphincter 20, which is a band of muscle at the junction between the pylorus 16 and the duodenum 18 of the small intestine. It will be appreciated that the pylorus 16 is a muscular valve between the stomach 14 and the small intestine, and the pyloric sphincter 20 periodically dilates to permit the stomach's contents to flow from the stomach 14 to the duodenum 18 and, thus, into the small intestine. That is, the pyloric sphincter 20 controls the flow of food, etc., from the stomach 14 to the small intestine. Generally, the pyloric sphincter 20 dilates every five to ten minutes, opening the passageway or flow path from the stomach 14 to the duodenum 18.

Usually, when inserting a catheter or cannula, such as a feeding tube, through the gastrointestinal tract such that the cannula passes through the stomach 14 and into the duodenum 20, a healthcare provider (such as a surgeon, physician, nurse, or the like) will wait for the pyloric sphincter 20 to dilate to insert or pass the cannula through the pyloric sphincter 20 and into the duodenum 18. However, such a placement technique can be challenging or difficult, as well as time consuming. Placing cannulas or the like through other sphincters can present the same challenges.

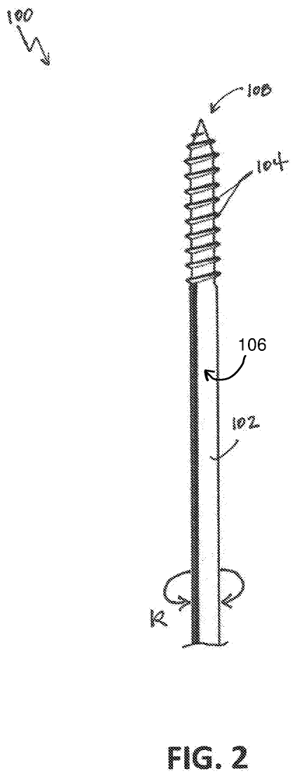

Referring now to FIGS. 2 through 5, the present subject matter provides a cannulation device including a threaded or screw feature, which may allow cannulation of a sphincter, such as the pyloric sphincter 20, without having to rely on natural dilation of the sphincter to open a passageway for the device. As illustrated in FIG. 2, the cannulation device 100 includes a cannula 102 with threads 104. In the exemplary embodiment of FIG. 2, the threads 104 are coupled to an outer surface 106 of the cannula 102 at or near a distal end 108 of the cannula 102, which is opposite a proximal end (not shown) of the cannula 102. In some embodiments, the threads 104 may be integral with the cannula 102 such that the cannulation device 100 is a single piece device, e.g., the threads 104 may be molded or otherwise formed with the cannula 102. In other embodiments, the threads 104 may be attached or otherwise coupled to the outer surface 106. The threads 104 may be formed from a relatively soft and/or flexible material, such as rubber or a material with a similar hardness as rubber, e.g., to help avoid irritation to the patient. More particularly, the entire threaded portion of the cannulation device 100 may be formed from the relatively soft and/or flexible material, e.g., to help avoid irritation and/or injury to the patient as the threaded portion is pressed and/or pushed against the patient's body as described herein. Further, any suitable number of threads 104 and any suitable pitch of the threads 104 may be used.

It will be appreciated that the threads 104 may be used as a screw or screw mechanism to induce dilation of a sphincter, such as a pyloric sphincter 20, such that the cannula 102 may be inserted through the sphincter. In some embodiments, when the distal end 108 is positioned at the sphincter, such that the threads 104 are located at the sphincter, a user (e.g., a healthcare provider such as a surgeon, physician, nurse, etc.) may manually rotate the cannulation device 100 while pressing or easing the distal end 108 against the sphincter. The rotational motion, shown schematically by arrows labeled R, helps engage the threads 104 with the sphincter to thereby induce dilation of the sphincter. As the sphincter dilates, the user may advance the cannulation device 100 through the sphincter, rotating the cannula 102 as needed to ensure adequate dilation of the sphincter. Similarly, the rotational motion may be reversed to reverse the direction of travel of the cannulation device 100 through the sphincter, e.g., to retract or remove the cannulation device 100 through the sphincter.

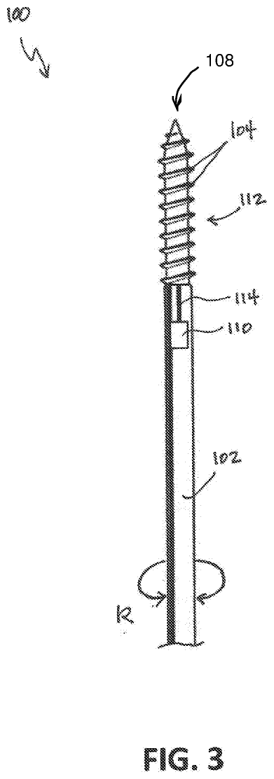

In other embodiments, instead of manually rotating the cannula, the user may activate an electric motor 110 in operative communication with the cannula 102 such that the electric motor 110 rotates the cannula 102 and, thereby, the threads 104 at the distal end 108 of the cannula 102. As illustrated in FIG. 3, the cannulation device 100 may include a threaded segment 112 at the distal end 108 of the cannula 102. The threaded segment 112 includes the threads 104 and may be separate from the cannula 102; that is, the threads 104 defining the screw portion of the cannulation device 100 may be separate from the cannula 102. Alternatively, as described above, the threads 104 may be attached or coupled to the cannula 102, or integrally formed with the cannula 102, such that the threaded segment 112 is part of the cannula 102 rather than separate from the cannula 102.

In some embodiments, only the threaded segment 112 is rotated, rather than the entire cannulation device 100. In other embodiments, however, the electric motor 110 may rotate the entire cannulation device 100, e.g., in a manner similar to manual rotation of the device 100. Thus, although the rotational motion of the threads 104 is illustrated schematically by the arrows labeled R that are away from the distal end 108 of the cannula, it will be appreciated that the arrows R are intended to show only the direction of rotation and are not intended to indicate which portion or portions of the cannulation device 100 are rotated, either manually or mechanically. Further, it will be understood that the cannulation device 100 may be rotated in one direction to advance the device 100 in along a generally linear path and may be rotated in the opposite direction to retract or remove the device 100, e.g., to reverse the direction of movement of the device 100 along the generally linear path.

As shown in the exemplary embodiment of FIG. 3, the electric motor 110 may be embedded in the cannulation device 100 near the threaded segment 112, e.g., the electric motor 110 may be embedded in the cannula 102 near its distal end 108. In some embodiments, the electric motor 110 may have a width within a range of one millimeter to ten millimeters (1 mm to 10 mm), e.g., the electric motor 110 may be two millimeters (2 mm) wide. A shaft 114 may extend from the electric motor 110 to the threaded segment 112 to operatively couple the electric motor 110 and the threads 104 and to translate the rotational motion generated by the motor 110 to the threads 104. In other embodiments, the electric motor 110 may be embedded at another location of the cannulation device 100, e.g., the motor 110 may be disposed at a location closer to or farther away from the threaded segment 112 than as shown in FIG. 3. Alternatively, the electric motor 110 may be separate from the cannula 102 and threaded segment 112, i.e., the motor 110 need not be embedded in any portion of the cannulation device 100.

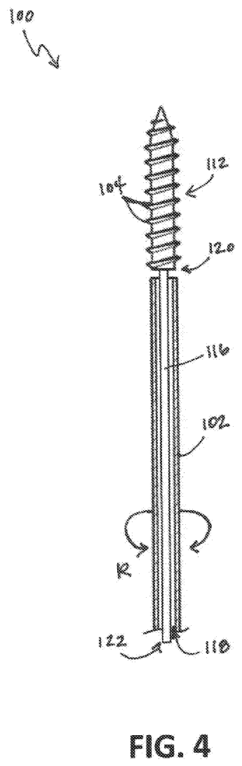

Referring now to FIG. 4, in some embodiments, the cannulation device 100 may include a stylet 116. As shown in FIG. 4, the threaded segment 112 may be attached or coupled to the stylet 116; in some embodiments, the threaded segment 112 may be integrally formed with the stylet 116. The stylet 116 may extend through a flow path 118 defined by the cannula 102 such that the threaded segment 112, which is attached to a distal end 120 of the stylet 116, extends from the distal end 108 of the cannula 102. The stylet 116 may be manually or mechanically manipulated to rotate the threaded segment 112 as described with respect to FIGS. 2 and 3. For example, the user may rotate a proximal end 122 of the stylet 116, e.g., directly or indirectly, such as via a component attached or secured to the stylet 116. That is, the stylet 116 may be configured to be manually rotated by a user of the cannulation device 100. In other embodiments, the stylet 116 may be coupled to an electric motor 110, which mechanically rotates the stylet 116 to rotate the threaded segment 112 coupled to the stylet 116. Further, once the sphincter has dilated and the cannulation device 100 inserted therethrough, the stylet 116 and the attached threaded segment 112 may be withdrawn through the cannula flow path 118. In other embodiments, the stylet 116 may be decoupled from the threaded segment 112 and only the stylet 116 may be removed through the cannula flow path 118. In such embodiments, the stylet 116 may be reinserted into the cannula flow path 118 and reconnected to the threaded segment 112, e.g., to enable repositioning or extraction of the cannulation device 100.

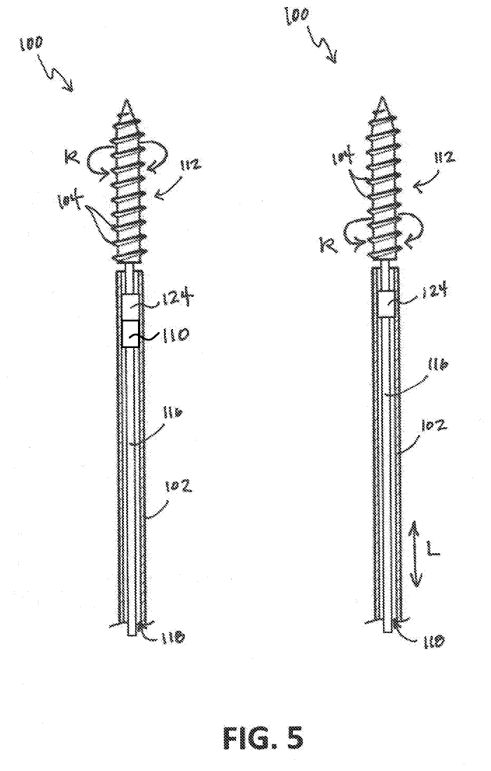

Turning to FIG. 5, other means for mechanically rotating the threads 104 may be used as well. For example, a gearbox 124 may be used in the rotation of the threads 104. In some embodiments, the gearbox 124 may be in operative communication with the electric motor 110, such that gears of the gearbox 124 step up or step down the torque provided by the electric motor 110 to rotate the threads 104 at an appropriate speed. That is, the gearbox 124 may adjust a rotational speed of the threads 104. In other embodiments, the gearbox 124 may be used to translate linear motion (indicated by the arrows labeled L) to rotational motion (indicated by the arrows labeled R), such that a linear input by a user or a mechanical device such as a motor may be converted to a rotational input to the threads 104 to rotate the threads 104. As shown in FIG. 5, the gearbox 124 may be operatively connected to the stylet 116, to which the threaded segment 112 may be attached or with which the threaded segment may be integrally formed, to rotate the threads 104. The gearbox 124 may be used in other ways as well, and other means for mechanically rotating the threads 104 also may be used.

Further, the screw mechanism or threaded segment 112 may help a user guide the cannulation device 100 within the patient's body. For instance, the user may tilt the threaded segment 112 in a desired direction to guide the cannula 102 in the desired direction. Thus, the threads 104 also may be used as a steering mechanism to control the direction of travel of the cannula 102 within the patient.

Additionally or alternatively, the threaded segment 112 may be useful for more than sphincter cannulation. For example, the threads 104, whether defined on the cannula or on a separate threaded segment 112, may be rotated to help propel the cannulation device 100 through the patient's body. More particularly, the threads 104 may be rotated manually or mechanically to propel the cannulation device 100 through the patient's body, such as through the patient's esophagus and/or stomach. In some embodiments, rather than the cannulation device 100 being pushed by a user (e.g., by pushing the stylet 116 inserted into the flow path 118), the rotation of the threads 104 may allow the device 100 to advance and/or retract itself through the patient. In other embodiments, the rotational motion of the threads 104 may be used together with pushing or other input by the user to advance and/or retract the cannula 102 with respect to the patient.

It will be appreciated that the cannulation device 110 may include a channel having an opening at each of the proximal end and distal end 108 of the cannula 102 to facilitate a flow of nutrients into the patient, i.e., to facilitate feeding the patient using the cannula 102. More particularly, the cannula flow path 118 may allow nutrients to flow therethrough, and the distal end 108 of the cannula 102 may be positioned within the patient's intestines, such as the duodenum 18, to allow the nutrients to be provided to the patient for sustenance. The openings at each of the proximal end and distal end 108 of the cannula 102 may allow the nutrients to enter and exit the cannula 102. Further, it will be understood that, in embodiments employing the stylet 116, the stylet 116 may be removed from the cannula flow path 118 to allow nutrients to flow through the flow path 118.

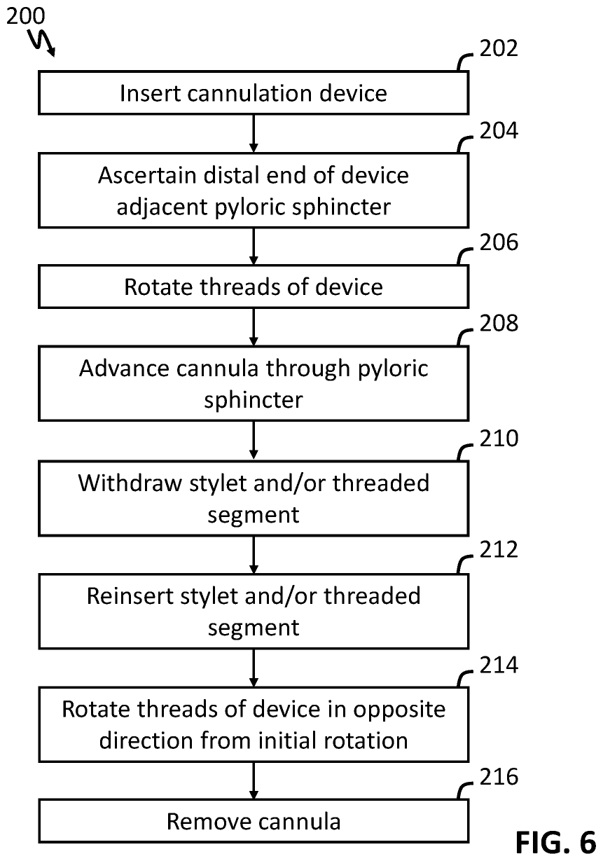

The present subject matter also provides methods for traversing a sphincter and, more particularly, methods for traversing a pyloric sphincter such as the pyloric sphincter 20 illustrated in FIG. 1. Referring to FIG. 6, a flow diagram is provided of an exemplary method 200 for cannulating the pyloric sphincter 20. As depicted at 202 in FIG. 6, the method 200 includes inserting the cannulation device 100 into the patient. For example, a user may insert the cannulation device 100 through the patient's nose or mouth and advance the device 100 down the patient's gastrointestinal tract 10, e.g., down the patient's esophagus 12 toward the patient's intestines, which are beyond the stomach 14, pylorus 16, and pyloric sphincter 20. As shown at 204, the user ascertains whether the distal end 108 of the cannulation device 100 is adjacent the pyloric sphincter 20. The position of the distal end 108 may be determined, e.g., based on the length of cannula 102 inserted into the patient or by other means, such as one or more sensors, tactile feedback from the cannulation device 100 interpreted by the user, etc.

Once the user ascertains that the distal end 108, where the threads 104 are disposed as described herein, is positioned at the pyloric sphincter 20, the threads 104 may be rotated to cannulate the sphincter 20, as shown at 206. That is, rotating the threads 104 into the pyloric sphincter 20, i.e., while the threads 104 are in contact with the pyloric sphincter 20, may create a type of screw action, through which the threads 104 (and thereby the cannula 102) are driven through the sphincter 20. As described herein, the threads 104 may be manually or mechanically rotated. For instance, the user may manually rotate the cannula 102 or a stylet 116 to which a threaded segment 112 is attached to drive or screw the cannula 102 through the pyloric sphincter 20. In other embodiments, an electric motor 110 and/or a gearbox 124 may mechanically rotate the threads 104 to drive or screw the cannula 102 through the pyloric sphincter 20.

As illustrated at 208 in FIG. 6, the method 200 includes advancing the cannula 102 through the pyloric sphincter 20 after screwing the threads 104 through the sphincter 20. In some embodiments, as shown at 210, the method 200 also may include withdrawing the threaded segment 112, which may be attached to a stylet 116 as described above. For example, when attached to the stylet 116, the threaded segment 112 may be withdrawn through the flow path 118 defined through the cannula 102. Alternatively, the threaded segment 112 may be detached or disconnected from the stylet 116 and only the stylet 116 withdrawn through the cannula flow path 118.

In some embodiments, the method 200 also may include withdrawing the cannula 102 from the patient. As shown at 212 in FIG. 6, in embodiments in which the stylet 116 was used to insert the cannula 102 and only the stylet 116 was withdrawn following cannulation, the stylet 116 may be reinserted into the cannula flow path 118 and reconnected to the threaded segment 112. Alternatively, in embodiments in which both the stylet 116 and threaded segment 112 were withdrawn following cannulation, both the stylet 116 and threaded segment 112 may be reinserted into the cannula flow path 118 and advanced to the distal end 108 of the cannula 102. As illustrated at 214, the threads 104 may be rotated, manually or mechanically, in a direction opposite to the direction in which the threads 104 were rotated to advance the cannula 102 into the patient to retract the cannula 102 through the patient, including through the pyloric sphincter 20. Then, as shown at 216 in FIG. 2, the cannulation device 100 may be removed from the patient.

The method 200 illustrated in FIG. 6 is provided by way of example only. It will be appreciated that the method 200 may be modified as needed for traversing any other sphincter or similar anatomical feature using the cannulation device 100. Further, as described herein, the method 200 may be modified as needed for maneuvering the cannulation device 100 through the patient's body, e.g., using the threads 104 to propel the cannulation device 100 through the patient's body.

Accordingly, the present subject matter provides a system and apparatus for cannulating sphincters such as pyloric sphincters. For example, the present subject matter provides a screw mechanism on a tip or distal end of a cannula (such as a feeding tube) to cannulate a pyloric sphincter. The threads of the screw mechanism may be rotated, e.g., manually or mechanically, against the pyloric sphincter to allow the pyloric sphincter to dilate sufficiently to insert the cannula. Such screw mechanism and methods of cannulation involving such screw mechanisms may simplify sphincter cannulation, e.g., by reducing or eliminating reliance on natural dilation of the sphincter, which may reduce challenges in sphincter cannulation, and/or by reducing the time required to cannulate a sphincter. Other benefits and advantages of the present subject matter also may be recognized by those of ordinary skill in the art.

It should also be appreciated that these procedures may involve treatment of humans by physicians, physician assistants, nurses, or other healthcare providers. In addition, these procedures may involve treatment of other mammals and animals by veterinarians, researchers, and others.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the present subject matter has been described in conjunction with specific embodiments thereof, it is evident that many alternatives, modifications, and variations will be apparent to those skilled in the art. Accordingly, it is intended to embrace all such alternatives, modifications, and variations that fall within the broad scope of the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims or if they include equivalent structural elements with insubstantial differences from the literal language of the claims.

1. A cannulation device, comprising:
a cannula for disposition within a patient's body, the cannula having a distal end; and
a threaded segment comprising threads, the threaded segment disposed at the distal end,
wherein the threaded segment comprises a tip of the cannulation device, and
wherein the tip is tapered.

2. The cannulation device of claim 1, wherein the threads are integral with the cannula.

3. (canceled)

4. The cannulation device of claim 1, further comprising:
an electric motor,
wherein the electric motor is operatively connected to the threads to rotate the threads.

5. The cannulation device of claim 4, wherein the electric motor is embedded in the cannula.

6. The cannulation device of claim 1, further comprising:
a threaded segment comprising the threads; and
an electric motor operatively connected to the threaded segment to rotate the threads.

7. The cannulation device of claim 1, further comprising:
a gearbox operatively connected to the threads,
wherein the gearbox translates linear motion to rotational motion to rotate the threads.

8. The cannulation device of claim 1, further comprising:
a gearbox operatively connected to the threads,
wherein the gearbox adjusts a rotational speed of the threads.

9. The cannulation device of claim 1, further comprising:
a threaded segment comprising the threads; and
a stylet extending through a flow path defined by the cannula,
wherein the threaded segment is attached to the stylet such that the threaded segment extends from the distal end of the cannula.

10. The cannulation device of claim 9, wherein the stylet is configured to be manually rotated by a user of the cannulation device to rotate the threads.

11. The cannulation device of claim 9, further comprising:
an electric motor,
wherein the electric motor is operatively connected to the stylet to rotate the threads.

12. The cannulation device of claim 9, further comprising:
a gearbox operatively connected to the stylet,
wherein the gearbox translates linear motion to rotational motion to rotate the threads.

13. The cannulation device of claim 9, further comprising:
a gearbox operatively connected to the stylet,
wherein the gearbox adjusts a rotational speed of the threads.

14. The cannulation device of claim 1, wherein the threads are formed from rubber.

15. The cannulation device of claim 1, wherein the threads are configured to be rotated when the threads are disposed adjacent a pyloric sphincter within the patient's body.

16. A pyloric sphincter cannulation device, comprising:
a cannula for positioning within an intestine of a patient's body, the cannula having a tapered distal end; and
threads disposed at the tapered distal end,
wherein the threads are configured to be rotated when the threads are disposed adjacent the patient's pyloric sphincter.

17. The pyloric sphincter cannulation device of claim 16, further comprising:
an electric motor,
wherein the electric motor is operatively connected to the threads to rotate the threads.

18. The pyloric sphincter cannulation device of claim 16, further comprising:
a threaded segment comprising the threads; and
a stylet extending through a flow path defined by the cannula,
wherein the threaded segment is attached to the stylet.

19. The pyloric sphincter cannulation device of claim 16, wherein the threads are integral with the cannula.

20. A method for traversing a pyloric sphincter of a patient, comprising:
inserting a cannulation device into the patient's gastrointestinal tract;
ascertaining a distal end of the cannulation device is adjacent the pyloric sphincter;
rotating threads of the cannulation device into the pyloric sphincter; and
advancing the cannulation device through the pyloric sphincter.

21. The cannulation device of claim 1, wherein the threads extend adjacent only the tip.
